(19) United States
(12) Patent Application Publication
(10) Pub. No.: US 2025/0388559 A1
(43) Pub. Date: Dec. 25, 2025

(54) PROCESS FOR THE SYNTHESIS OF PROTOCATECHUIC ALDEHYDE METHYLENE ETHER

(71) Applicant: Sachin SONAVANE, Navi Mumbai (IN)

(72) Inventor: Sachin SONAVANE, Navi Mumbai (IN)

(21) Appl. No.: 19/243,121

(22) Filed: Jun. 19, 2025

(30) Foreign Application Priority Data
Jun. 22, 2024 (IN) 202421048067

(51) Int. Cl. C07D 317/54; B01J 31/02; C07D 317/52

(52) U.S. Cl. CPC C07D 317/54; B01J 31/0222; B01J 31/0225; C07D 317/52

(57) ABSTRACT

The present invention relates an improved process for the preparation of piperonal (2), which is useful as an intermediate for the preparation of Tadalafil of formula (1).

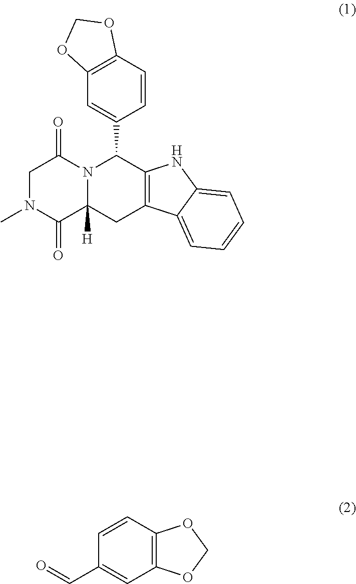 [SMILES: CN1CC(=O)N2[C@H](c3ccc4c(c3)OCO4)c3[nH]c4ccccc4c3C[C@@H]2C1=O ; O=Cc1ccc2c(c1)OCO2]

### CROSS-REFERENCE TO RELATED APPLICATIONS

This application claims priority under 35 U.S.C. § 119 to patent application No. 202421048067, filed on Jun. 22, 2024 in India, the disclosure of which is incorporated herein by reference it its entirety.

### FIELD OF THE INVENTION

The present invention relates an improved process for the preparation of piperonal (2), which is useful as an intermediate for the preparation of Tadalafil of formula (1).

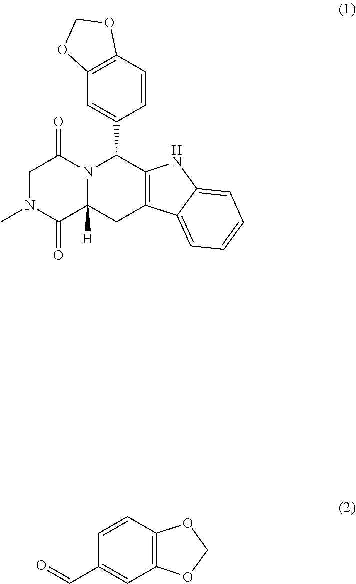 [SMILES: CN1CC(=O)N2[C@H](c3ccc4c(c3)OCO4)c3[nH]c4ccccc4c3C[C@@H]2C1=O ; O=Cc1ccc2c(c1)OCO2]

### BACKGROUND OF THE INVENTION

The following discussion of the prior art is intended to present the invention in an appropriate technical context and allow its significance to be properly appreciated. Unless clearly indicated to the contrary, reference to any prior art in this specification should not be construed as an expressed or implied admission that such art is widely known or forms part of common general knowledge in the field.

Tadalafil is a potent and selective inhibitor of the cyclic guanosine monophosphate (cGMP)-specific phosphodiesterase enzyme, PDE5. The inhibition of PDE5 increases the amount of cGMP, resulting in smooth muscle relaxation and increased blood flow.

Piperonal is used as a key intermediate to manufacture APIs like Tadalafil, Droxidopa and Stiripentol. Piperonal is also known as heliotropin.

There are various chemical methods disclosed in the prior art for the synthesis of Piperonal.

Japanese patent application 156867/77 discloses a process for the preparation of piperonal, comprising formylation of methylenedioxybenzene using N-methyl formanilide. However, about 60% of 1,2-methylenedioxybenzene is recovered, and the energy consumption is very high, and therefore this is not advantageous as an industrial process.

J. Gen. Chem 8, 975 (1938) and British Pat. 1538214 disclose chloromethylation process for the preparation of heliotropin comprising chloromethylation of methylenedioxybenzene to obtain 3,4-methylenedioxybenzyl chloride which in turn is reacted with alkali metal salt of 2-nitropropane to obtain heliotropin. The yield of end product is 55-60% based on methylenedioxybenzene and also a large amount of tar like material is formed as a byproduct.

U.S. Pat. No. 10,633,360 B2 discloses a process for the preparation of heliotropin comprising reacting methylenedioxybenzene with formaldehyde and HCl to obtain methylenedioxybenzyl chloride, which is isolated and reacted with hexamethylenetetramine using toluene as a solvent to obtain heliotropin as shown in Scheme 1.

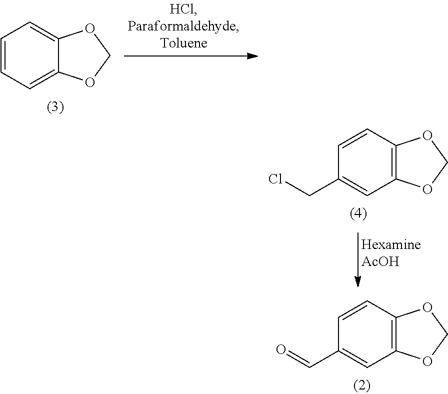 [SMILES: c1ccc2c(c1)OCO2 ; ClCc1ccc2c(c1)OCO2 ; O=Cc1ccc2c(c1)OCO2]

U.S. Pat. No. 11,319,297 B2 claims a process of preparation of methylenedioxybenzyl chloride comprising reacting methylenedioxybenzene with a chloromethylating agent generated in-situ by the reaction of a formaldehyde precursor and hydrogen chloride, in a solvent and in the presence of a catalytic amount of a carboxylic acid. The carboxylic acid used is selected from acetic acid, propionic acid, formic acid or hexanoic acid.

WO2008023836 discloses a process for the preparation of piperonal comprising reacting methylenedioxybenzene with formaldehyde and aqueous HCl in toluene to obtain piperonyl chloride in 85% yield based on internal online GC standard without mention of yield of isolated material. The piperonyl chloride formed in first part is reacted with acetic acid and hexamethylenetetramine in molar ratio of 0.25 moles to 1.0 mole per mole of piperonyl chloride to obtain the complex which is subsequently decomposed to obtain piperonal. The piperonal obtained is isolated by extraction with ethyl acetate. The process disclosed therein involves the use of multiple solvents, as well as isolation/purification at intermediate stages. Moreover, the purity of the final product obtained is not mentioned.

CN117143070A directed the process of preparation of heliotropin comprising steps of: which is characterized by comprising the following steps: step 1, mixing glyoxylic acid-sulfuric acid-methanesulfonic acid, and cooling to a certain temperature in advance; step 2, dropwise adding piper nigrum ring, and reacting under the temperature control condition by taking sulfuric acid-methanesulfonic acid as a catalyst; step 3, washing and drying to obtain a 3, 4-methylenedioxybenzilic acid intermediate; and 4, performing oxidative decarboxylation on the 3, 4-methylenedioxybenzilic acid intermediate by using a sulfuric acid-chromatographic silica gel nitric acid oxidation system, and performing extraction, concentration, ethanol crystallization and recrystallization purification on the intermediate to obtain the high-purity piperonal.

Many prior arts directed the process for the preparation of piperonal. However, there is still a need for a process that can avoid the high temperature and pressure with easy work up of the reaction to get the intermediate in pure form without extra purification, which is responsible for yield loss. The object of the present invention is to provide a solution to the technical problems associated with the prior art. This instant invention mainly directed the process, which is environment friendly without any hazardous solvent and reaction conditions. Also, this invention disclosed the process which is economically and commercially viable.

The present invention relates to a process for the preparation of piperonal of formula (2), comprising:
a) reacting methylenedioxybenzene (3) with paraformaldehyde and hydrogen chloride in a solvent and in presence of a catalytic amount of a p-Toluene sulfonic acid (PTSA) to give piperonyl chloride (4); and

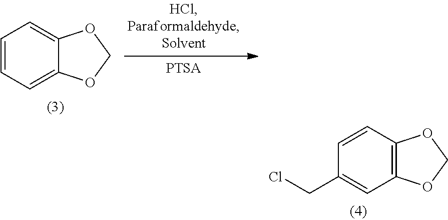 [SMILES: c1ccc2c(c1)OCO2 ; ClCc1ccc2c(c1)OCO2]

b) reacting piperonyl chloride (4) with hexamethylenetetramine in a solvent and in presence of glacial acetic acid, which is after subsequent hydrolysis (Sommelet reaction conditions) or after base catalyzed hydrolysis to give piperonal (2).

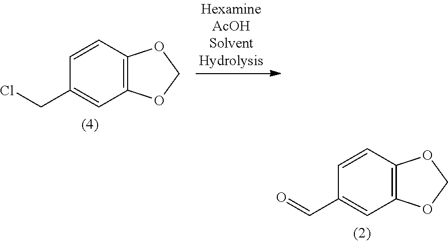 [SMILES: ClCc1ccc2c(c1)OCO2 ; O=Cc1ccc2c(c1)OCO2]

### SUMMARY OF THE INVENTION

The present invention is directed to an improved process for the preparation of piperonal and the objectives of the invention are provided below.

It is an object of the present invention is to provide an improved, and industrially feasible, economical viable process for preparing piperonal (2).

Another object of the present invention is to provide an improved process for the preparation of piperonal (2) that is simple and convenient without any additional purification of the intermediate stages.

Another aspect of the present invention is that piperonyl chloride is isolated in pure form by distillation to avoid the byproduct formation during subsequent step and eventually produce high grade piperonal with purity level above 99.5%. This also hereby reduces the unit operations, minimizing handling losses and improving overall efficiency of the process

A further object of the present invention is to disclose a process for chloromethylation of substituted benzenes, eliminating the use of Lewis acid catalysts and/or strong acids like halo sulphonic acid, sulfuric acid, thereby eliminating or minimizing generation of effluent typically associated with conventional chloromethylation processes.

More particularly the present invention relates to a improved process for the preparation of piperonal of formula (2), comprising the steps of:
a) reacting methylenedioxybenzene (3) with paraformaldehyde and hydrogen chloride in a solvent and in presence of a catalytic amount of a catalyst to give piperonyl chloride (4); and

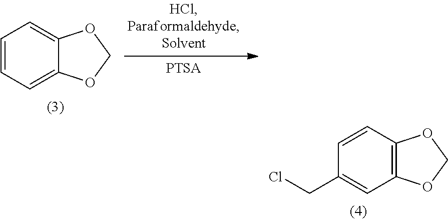 [SMILES: c1ccc2c(c1)OCO2 ; ClCc1ccc2c(c1)OCO2]

b) reacting piperonyl chloride (4) with hexamethylenetetramine in a solvent and in presence of glacial acetic acid, which is after subsequent hydrolysis (Sommelet reaction conditions) or after base catalysed hydrolysis to give piperonal (2).

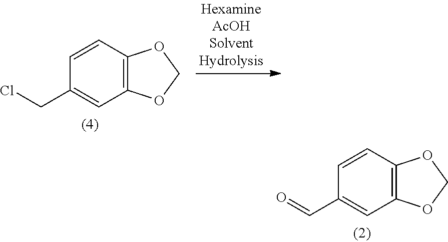 [SMILES: ClCc1ccc2c(c1)OCO2 ; O=Cc1ccc2c(c1)OCO2]

### DETAILED DESCRIPTION OF THE INVENTION

Before the present invention is described, it is to be understood that this invention is not limited to particular methodologies and materials described, as these may vary as per the person skilled in the art. It is also to be understood that the terminology used in the description is for the purpose of describing the particular embodiments only and is not intended to limit the scope of the present invention.

Before the present invention is described, it is to be understood that unless defined otherwise, all technical and scientific terms used herein have the same meaning as commonly understood by one of ordinary skill in the art to which this invention belongs. Further, it is to be understood that the present invention is not limited to the methodologies and materials similar or equivalent to those described herein that can be used in the practice or testing of the present invention, the preferred methods and materials are described, as these may vary within the specification indicated. Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive but may be implemented in various combinations. The described embodiments of the invention and the disclosed examples are given for the purpose of illustration rather than limitation of the invention as set forth the appended claims. Further, the terms disclosed in embodiments are merely exemplary methods of the invention, which may be embodied in various forms.

The term “about,” as used herein, is intended to qualify the numerical values, which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “heating”, as used herein, is heating the solution gradually to a temperature in the range of 50-150° C.

The present invention relates to a process for the preparation of piperonal of formula (2), comprising:
a) reacting methylenedioxybenzene (3) with paraformaldehyde and hydrogen chloride in a solvent and in presence of a catalytic amount of catalyst to give piperonyl chloride (4); and

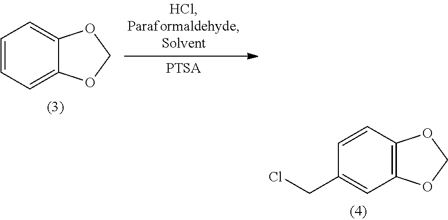 [SMILES: c1ccc2c(c1)OCO2 ; ClCc1ccc2c(c1)OCO2]

b) reacting piperonyl chloride (4) with hexamethylenetetramine in a solvent and in presence of glacial acetic acid, which is after subsequent hydrolysis (Sommelet reaction conditions) or after base catalysed hydrolysis to give piperonal (2).

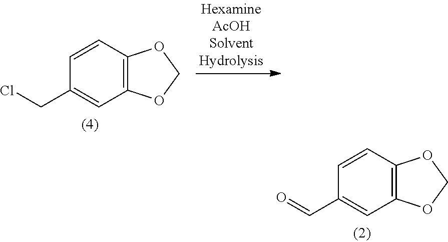 [SMILES: ClCc1ccc2c(c1)OCO2 ; O=Cc1ccc2c(c1)OCO2]

The solvent used in step (a) and step (b) is an ether solvent selected from tetrahydrofuran, cyclopentyl methyl ether, 2-methyltetrahydrofuran, diethyl ether, dioxane, 1,4-dioxane, 1,2-dioxane or 1,3-dioxane; an alcoholic solvent selected from methanol, ethanol, isopropanol, t-amyl alcohol, t-butyl alcohol or hexanol; an ester solvent selected from ethyl acetate, methyl acetate, isopropyl acetate, hexyl acetate, butyl acetate, sec-butyl acetate or tert-butyl acetate; a halogenated solvent selected from dichloromethane (DCM), 4-bromotoluene, diiodomethane, carbon tetrachloride, chlorobenzene or chloroform; a ketone solvent selected form acetone, propanone, methyl ethyl ketone or methyl isobutyl ketone; an aprotic solvent selected from acetonitrile, N,N-dimethyl formamide (DMF), N,N-dimethyl acetamide, dimethyl sulfoxide (DMSO) or N-methylpyrrolidone (NMP); an aromatic solvent selected from toluene, xylene or benzene; water or a mixture thereof.

The catalyst used in step (a) in chloromethylation is selected from Benzene sulphonic acid, methyl sulphonic acid, Para Toluene sulphonyl chloride, methane sulphonyl chloride, and Para toluene sulphonic acid but preferably is used Para toluene sulphonic acid.

According to this instant invention - - - a chloromethylated compound of formula (4) (also referred as piperonyl chloride) is obtained in substantially pure form and high yield, from the corresponding compound of formula (3). The process comprises contacting the compound of formula (3) with a chloromethylating agent generated in-situ by reaction of paraformaldehyde and hydrogen chloride, in a suitable solvent/contacting medium and in presence of a catalytic amount of para toluene sulfonic acid (PTSA). Para toluene sulfonic acid acts as a promotor for the said chloromethylation reaction and thereby eliminating the use of any Lewis acids such as aluminium chloride, zinc chloride and the like, or organic and inorganic acids such as carboxylic acid e.g. acetic acid and or sulphuric acid and the like, as reported in the prior art, which has the benefit of significantly reducing the effluent generated and making the process environmentally friendly.

The inventors of this instant invention arrived at the combination of solvent catalysts and chloromethylating agents with suitable reaction conditions through rigorous experimentation. To prove the importance of selective catalysts, the example section provided an example without the use of a catalyst, wherein the yield is obtained only 53%.

In the present invention, the reaction conditions such as reaction temperature, choice of solvent and para toluene sulfonic acid in catalytic amount used as reaction promoter, played vital role to obtain a high yield of the corresponding chloromethylated product of formula (4). As mentioned above, compounds of formula (4) is converted to the compound of formula (2) using the Sommelet reaction. However, the conversion may also be carried out using base catalyzed hydrolysis.

The compound of Formula (4) is isolated by distillation process, which further contacted with hexamethylenetetramine at about 100° C. preferably at about 80° C. to obtain a complex is added an acid or base or salt selected from the group comprising aqueous acetic acid, or a mixture of aqueous acetic acid and mineral acids selected from the group comprising sulfuric acid, hydrochloric acid, nitric acid or mixtures thereof, or a mixture of organic acids selected from the group comprising acetic acid and chloroacetic acid and the likes, or a mixture of aqueous acetic acid and alkali metal salts selected from the group comprising sodium acetate or sodium phosphates and the likes or mixture thereof, or some combination of the above to obtain a biphasic reaction mass; the said biphasic reaction mass is maintained in the acidic range of pH of about 2 to about 6 during reaction, and the said biphasic reaction mass is digested comprising heating at about 500° C. to about 110° C., preferably at about 80° C. to about 90° C. for about 15 hours preferably about 8 hours, so as to decompose the complex formed to produce the corresponding alkoxy substituted benzaldehyde of Formula (2).

In biphasic system, organic solvent phase containing compound of Formula (2) is separated from the aqueous Phase by layer separation. If required the aqueous phase can be further extracted with the same organic solvent. The organic solvent extracts are combined and solvent is recovered to obtain crude product, which is purified by high vacuum distillation.

The process of the present invention uses an inorganic base instead of an organic base. Therefore, disposing of the base in the work-up stages is very simple and more environmentally friendly. Preferably, the inorganic base is selected from the group consisting of a metal hydroxide and a metal carbonate. Preferably, the metal hydroxide is sodium hydroxide, potassium hydroxide, cesium hydroxide, barium hydroxide, magnesium hydroxide, calcium hydroxide or strontium hydroxide. Preferably, the metal carbonate is sodium carbonate, sodium bicarbonate, potassium carbonate, potassium bicarbonate or cesium carbonate.

The inventors of the present invention have observed that chloromethylation of formula (2) by the reaction with the chloromethylating agent generated in-situ is accelerated in the presence of para toluene sulphonic acid present in catalytic quantities. Although the prior art discloses use of anhydrous mixture of zinc chloride in combination with low molecular weight carboxylic acid like acetic or propionic acid is extremely active in promoting the chloromethylation, the ratio of zinc chloride and carboxylic acid used was very high, typically in the range 1:2 to 4 moles of zinc chloride to carboxylic acid). Moreover, none of the prior art teaches the use of para toluene sulphonic acid, present in catalytic quantities acting as catalyst/promoter to accelerate the chloromethylation reaction.

According to another embodiment of the present invention, the work-up of step (a) further comprises the steps of:
(i) separating both organic and aqueous layer;
(ii) concentrating the layer to obtain the compound of formula (4) and
(iii) Isolating the compound formula (4) in pure form.

According to another embodiment of the present invention, the work-up of step (b) further comprises the steps of:
(i) separating both organic and aqueous layer;
(ii) washing aqueous layer with same organic solvent;
(iii) washing the combine organic layer with water;
(iv) concentrating the organic layer to obtain the compound of formula (2).

The details synthetic scheme of preparation of piperonal of formula (2) according to the present invention can be represented as below:

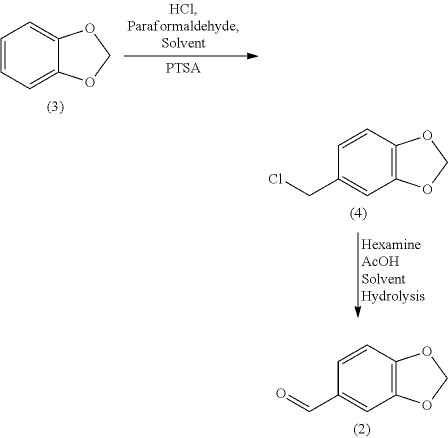 [SMILES: c1ccc2c(c1)OCO2 ; ClCc1ccc2c(c1)OCO2 ; O=Cc1ccc2c(c1)OCO2]

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example-1

### Preparation of Piperonyl Chloride Compound Formula (4)

A 500 ml three-necked round-bottomed flask equipped with a mechanical stirrer with teflon paddle and ice-bath. 111 g aqueous hydrochloric acid (30-35%) was charged in the flask. The stirring was started with a slow stream of HCl gas passing through the mixture. 1.5 g PTSA (catalytic amount), 97 g paraformaldehyde (purity NLT 92%) were combined in the flask. Cooled the reaction mixture to around 10° C. and stirred at the same temperature. 122 g Methylenedioxybenzene solution in 50 ml toluene was charged in flask around 10° C. The reaction mixture was stirred and concentration of HCl was maintained. Reaction progress was monitored by GC/TLC. When the reaction was complete, the aqueous and organic layers were separated. Distilled off the toluene completely from the organic layer under reduced the pure Piperonyl chloride as pressure and obtained pure Piperonyl chloride as a clear yellow liquid.
Yield: 88-91%;
Boiling Point: Between 94-100° C.
Density: Between 1.28-1.35
   Note: A various catalyst is used to perform the reaction like Benzene sulphonic acid, methyl sulphonic acid, Para Toluene sulphonyl chloride, methane sulphonyl chloride, and Para toluene sulphonic acid.

### Example-2

Preparation of Piperonyl Chloride (4) without Catalyst

A 500 ml three-necked round-bottomed flask equipped with a mechanical stirrer with teflon paddle and ice-bath. 111 g aqueous hydrochloric acid (30-35%) was charged in the flask. The stirring was started with a slow stream of HCl gas passing through the mixture. 97 g paraformaldehyde (purity NLT 92%) were combined in the flask. Cooled the reaction mixture to around 10° C. and stirred at the same temperature. 122 g Methylenedioxybenzene solution in 50 ml toluene was charged in flask around 10° C. The reaction mixture was stirred and concentration of HCl was maintained. Reaction progress was monitored by GC/TLC. Online conversion is 60% only. The aqueous and organic layers were separated. Distilled off the toluene completely from the organic layer under reduced pressure to isolate the pure Piperonyl chloride as clear yellow liquid.
Yield: 53%;
Boiling Point: Between 94-100° C.
Density: Between 1.28-1.35

### Example-3

### Preparation of Piperonol (2)

A 250 ml three-necked round-bottomed flask equipped with a mechanical stirrer with teflon paddle and ice-bath. 90 g Piperonyl chloride, 100 ml toluene, 150 g hexamethylenetetramine, were combined and stirred in the flask at 90° C. Monitor progress of the reaction on GC/TLC. If complies, add 100 g acetic acid solution to the reaction mass and solution was heated to 90° C. and stirred for 120 minutes at the same temperature for 8 hrs. pH of reaction mixture should be less than 2 to 5. When the reaction was complete, the organic and aqueous layers were separated, and aqueous layer was extracted with toluene. Combined the organic layers and washed with water. Distilled off the toluene completely from the organic layer under reduced pressure to get the pure piperonal.
Yield: 88%;
Purity: 99.5%
Melting Point: Between 35-38° C.

1. A method of preparing piperonal represented by formula (2), the method comprising:
a) reacting methylenedioxybenzene, represented by formula (3), with paraformaldehyde and hydrogen chloride in a solvent and in the presence of a catalyst to form piperonyl chloride, represented by formula (4);
b) isolating the piperonyl chloride by distillation to remove process impurities; and 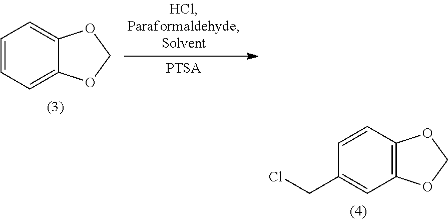 [SMILES: c1ccc2c(c1)OCO2 ; ClCc1ccc2c(c1)OCO2]
c) reacting the isolated piperonyl chloride with hexamethylenetetramine in a solvent and hydrolyzing with acetic acid to form piperonal.

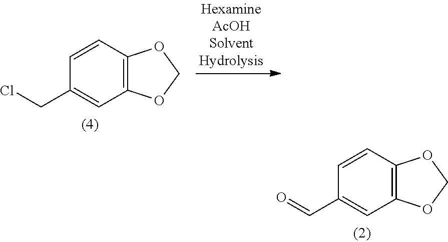 [SMILES: ClCc1ccc2c(c1)OCO2 ; O=Cc1ccc2c(c1)OCO2]

2. The method of claim 1, wherein the solvent used in step (a) is selected from the group consisting of tetrahydrofuran, cyclopentyl methyl ether, 2-methyltetrahydrofuran, diethyl ether, dioxane, 1,4-dioxane, 1,2-dioxane, 1,3-dioxane, methanol, ethanol, isopropanol, t-amyl alcohol, t-butyl alcohol, hexanol, ethyl acetate, methyl acetate, isopropyl acetate, hexyl acetate, butyl acetate, sec-butyl acetate, tert-butyl acetate, dichloromethane (DCM), 4-bromotoluene, diiodomethane, carbon tetrachloride, chlorobenzene, chloroform, acetone, propanone, methyl ethyl ketone, methyl isobutyl ketone, acetonitrile, N,N-dimethylformamide (DMF), N,N-dimethylacetamide, dimethyl sulfoxide (DMSO), N-methylpyrrolidone (NMP), toluene, xylene, benzene, water, or combinations thereof.

3. The method of claim 1, wherein the catalyst used in step (a) is selected from the group consisting of benzenesulfonic acid, methylsulfonic acid, para-toluenesulfonyl chloride, methanesulfonyl chloride, and para-toluenesulfonic acid.

4. The method of claim 1, wherein the solvent used in steps (b) and (c) is selected from the group consisting of tetrahydrofuran, cyclopentyl methyl ether, 2-methyltetrahydrofuran, diethyl ether, dioxane, 1,4-dioxane, 1,2-dioxane, 1,3-dioxane, methanol, ethanol, isopropanol, t-amyl alcohol, t-butyl alcohol, hexanol, ethyl acetate, methyl acetate, isopropyl acetate, hexyl acetate, butyl acetate, sec-butyl acetate, tert-butyl acetate, dichloromethane (DCM), 4-bromotoluene, diiodomethane, carbon tetrachloride, chlorobenzene, chloroform, acetone, propanone, methyl ethyl ketone, methyl isobutyl ketone, acetonitrile, N,N-dimethylformamide (DMF), N,N-dimethylacetamide, dimethyl sulfoxide (DMSO), N-methylpyrrolidone (NMP), toluene, xylene, benzene, water, or combinations thereof.

5. The method of claim 1, wherein step (c) further comprises adding an inorganic base selected from the group consisting of sodium hydroxide, potassium hydroxide, cesium hydroxide, barium hydroxide, magnesium hydroxide, calcium hydroxide, strontium hydroxide, sodium carbonate, sodium bicarbonate, potassium carbonate, potassium bicarbonate, or cesium carbonate.

6. The method of claim 1, wherein the isolating the piperonyl chloride in step (b) comprises
separating an organic layer and an aqueous layer; and
concentrating the organic layer to obtain piperonyl chloride, represented by formula (4).

7. The method of claim 1, wherein the work-up of step (c) comprises
separating an organic layer and an aqueous layer;
washing the organic layer with water; and
concentrating the organic layer to obtain piperonal represented by formula (2).
